(19) United States
(12) Patent Application Publication
(10) Pub. No.: US 2013/0171669 A1
(43) Pub. Date: Jul. 4, 2013

(54) POROUS MEMBRANES HAVING A HYDROPHILIC COATING AND METHODS FOR THEIR PREPARATION AND USE

(71) Applicants: Bing Li, Troy, NY (US); David Roger Moore, Rexford, NY (US)

(72) Inventors: Bing Li, Troy, NY (US); David Roger Moore, Rexford, NY (US)

(73) Assignee: GENERAL ELECTRIC COMPANY, SCHENECTADY, NY (US)

(21) Appl. No.: 13/340,793

(22) Filed: Dec. 30, 2011

(51) Int. Cl. G01N 33/566; C07K 16/00; B32B 23/00; B32B 3/26; B32B 23/08; B32B 27/06; B01J 19/12; C08B 15/06; C07K 14/00

(52) U.S. Cl. 435/7.92; 428/532; 428/508; 428/479.3; 428/315.7; 436/501; 435/7.9; 204/157.64

(57) ABSTRACT

A modified porous membrane comprising a polymeric hydrophilic coating bonded to a porous membrane is described. The polymeric hydrophilic coatings grafted to the porous membranes comprise, for example, a PEG moiety such as a PEGMA, a PEGDA, or a TMPET, wherein the polymeric hydrophilic coating on the porous membrane decreases non-specific binding of unwanted material to the porous membrane and increases the signal to noise ratio in immunoassays, in vitro diagnostic tests, and point of care tests. Methods of making these modified porous membranes are also disclosed.

### FIELD OF THE INVENTION

The present disclosure generally relates to porous membranes permanently grafted with a hydrophilic coating to minimize non-specific binding to porous membranes that are used for the immobilization of one or more specific biomolecules on the porous membrane for further analysis. Methods of preparing and using the modified porous membranes with these hydrophilic coatings are also described.

### BACKGROUND

Porous membranes, such as nitrocellulose membranes, are routinely used in a variety of processes, including biological applications that require the immobilization of one or more biomolecules. These biomolecules include but are not limited to proteins (e.g., antibodies) and nucleic acids (e.g., deoxyribonucleic acid (DNA) and ribonucleic acid (RNA)). Membranes able to both immobilize specific biomolecules of interest while at the same time minimizing non-specific binding of various molecules that interfere with the performance of, for example, immunoassays, in vitro diagnostic tests, particularly point-of-care diagnostic methods, and separation of analytes or biomolecules in biological samples (e.g., blood, urine, saliva, sputum, other bodily secretions, cells, and tissue samples) are desirable in the art. Such membranes would find use in a variety of biological processes and medical techniques.

Nitrocellulose membranes exhibit an essentially non-specific interaction between the nitrocellulose membrane and biomolecule(s). Researchers have traditionally relied upon this passive association as the basis for the use of nitrocellulose membranes in a variety of “entrapment” type immobilization methods. Reliance on this passive interaction between a nitrocellulose membrane and a biomolecule of interest, however, leads to complications for successfully using nitrocellulose membranes in many biological applications. This technique necessarily limits the amount of the biomolecule that can be immobilized on the nitrocellulose membrane and, equally problematic, also permits non-specific binding of undesirable molecules (e.g., not the biomolecule of interest) to the nitrocellulose membrane. Reducing non-specific binding would allow for an increase in specific binding of the biomolecule of interest to the nitrocellulose membrane and also a decrease in the signal (e.g., desired binding of the desired biomolecule to the nitrocellulose membrane) to noise (e.g., non-specific binding of unwanted material to the nitrocellulose) ratio. Decreasing the signal to noise ratio would increase the performance and sensitivity of, for example, immunoassays, in vitro diagnostic tests, particularly point-of-care diagnostic methods, and separation methods of analytes or biomolecules from other materials in biological samples (e.g., blood, lymph, urine, saliva, sputum, other bodily secretions, cells, and tissue samples). A reduction in the signal to noise ratio in, for example, immunoassays is desirable in the art.

New compositions and methods of modifying (e.g., chemically modifying) porous membranes to improve immobilization and binding of biomolecules (e.g., proteins and nucleic acids) of interest to porous membrane substrates are needed in the art. Such modified porous membranes, including modified nitrocellulose membranes, would find use in, for example, immunoassays, in vitro diagnostic tests (e.g., point-of-care diagnostic applications), and techniques for the separation of biomolecules of interest in biological samples. Porous membranes, more particularly nitrocellulose membranes, coated with a compound to decrease non-specific binding to the membrane are needed in the art. Such membranes would improve the performance and sensitivity of, for example, numerous immunoassays.

New methods of modifying (e.g., chemically modifying) porous membranes (e.g., nitrocellulose membranes) to decrease non-specific binding of unwanted material to porous membrane substrates are needed in the art. Porous membranes, more particularly nitrocellulose membranes, coated with a compound to decrease non-specific binding to the membrane would be advantageous. Such membranes would improve the performance and sensitivity of, for example, numerous immunoassays by decreasing non-specific binding to the membranes, by potentially eliminating the need for traditional blocking agents used in, for example, immunoassays to minimize non-specific binding, and by increasing the signal to noise ratio relative to that observed in immunoassays performed with unmodified porous (e.g., nitrocellulose membranes).

### BRIEF DESCRIPTION

Modified porous membranes, particularly nitrocellulose membranes, are described herein. In a particular embodiment, a nitrocellulose membrane comprises a polymeric hydrophilic coating bonded to the nitrocellulose membrane. The polymeric hydrophilic coating is generally permanently (e.g., covalently) bonded to the nitrocellulose membrane. The polymeric hydrophilic coating may be bonded to the nitrocellulose membrane by any method, including by exposure to electron beam (e-beam) irradiation of the porous (e.g., nitrocellulose) membrane.

The compositions herein include a modified porous membrane such as a nitrocellulose membrane that comprises a polymeric hydrophilic coating typically permanently bonded to the membrane. The compositions find use in methods that rely on the binding of one or more biomolecule(s), such as proteins (e.g., antibodies) and nucleic acids (e.g., DNA or RNA), to porous membranes, including but not limited to, nitrocellulose membranes. In particular aspects, the compositions are utilized in immunoassays, in vitro diagnostic tests, techniques for the identification or isolation of biomolecules of interest from biological samples (e.g., blood, urine, saliva, sputum, and samplings of cells or tissues), and various other biological methods that require the immobilization of a biomolecule on a porous membrane substrate like a nitrocellulose membrane. The porous membranes, particularly nitrocellulose membranes, comprise a polymeric hydrophilic coating bonded to the membrane, wherein the membrane decreases non-specific binding of unwanted material to the porous membrane, more particularly a nitrocellulose membrane. The polymeric hydrophilic coating on the nitrocellulose membranes described herein may include, but is not limited to, a polyethylene glycol (PEG) moiety, a polyvinyl alcohol, a hydroxyl group, a negatively charged ionic group, a positively charged ionic group, a zwitterionic group, or any combination thereof.

### DRAWINGS

These and other features, aspects, and advantages of the chemically modified porous membranes will become better understood when the following detailed description is read with reference to the accompanying drawings in which like characters represent like parts throughout the drawings, wherein:

FIG. 1 is a schematic representation of the mechanism of a polymeric hydrophilic coating on a nitrocellulose membrane by e-beam irradiation.

### DETAILED DESCRIPTION

Modified porous membranes, particularly nitrocellulose membranes, are provided herein that comprise at least one polymeric hydrophilic coating bonded to the porous membrane (e.g., nitrocellulose membrane). The porous membranes, such as nitrocellulose membranes, are traditionally used to immobilize a biomolecule (e.g., DNA, RNA, or protein) on a porous solid substrate. The term “modified” as used herein, particularly in reference to the disclosed porous membranes (e.g., nitrocellulose membranes) is intended to include any alteration to the membrane, for example, a chemical alteration, of the original, unmodified membrane. The “modified” porous membranes (e.g., nitrocellulose membranes) of the invention may be a nitrocellulose membrane comprising a polymeric hydrophilic coating bonded to the nitrocellulose membrane. The hydrophilic coating comprises, for example, a polyethylene glycol moiety, a polyvinyl alcohol, a hydroxyl group, a negatively charged ionic group, a positively charged ionic group, a zwitterionic group, or any combination thereof. In certain aspects, the polymeric hydrophilic coating comprises a PEG moiety, such as a PEGMA, a PEGDA, or a TMPET. PEG moieties of all molecular weights are encompassed by the instant disclosure.

To more clearly and concisely describe and point out the subject matter of the claimed invention, the following definitions are provided for specific terms, which are used in the following description and in the appended claims. Throughout the specification, exemplification of specific terms should be considered as non-limiting examples.

The term “non-specific binding” as used herein refers to the attachment of a biomolecule on a porous membrane (e.g., a nitrocellulose membrane) that is occurs by passive interaction of the biomolecule and the membrane and is independent of any particular, active interaction between the biomolecule and the membrane. “Non-specific binding” is also often referred to as “background binding” to a porous membrane, particularly a nitrocellulose membrane. One example of non-specific binding includes the attachment of a DNA molecule to a nitrocellulose membrane merely resulting from a random encounter in solution.

The term “modified” as used herein, particularly in reference to the disclosed porous membranes (e.g., nitrocellulose membranes), is intended to include any alteration to the membrane, for example, a chemical alteration, of the original, unmodified porous membrane (e.g., nitrocellulose membrane) substrate. The “modified” porous membranes, more particularly nitrocellulose membranes, set forth herein may be modified (e.g., chemically modified) nitrocellulose membranes comprising a polymeric hydrophilic coating bonded to the nitrocellulose membrane. The polymeric hydrophilic coating in particular embodiments comprises a PEG moiety, including but not limited to, a PEGMA, a PEGDA, or a TMPET.

Methods for preparing the porous membranes (e.g., nitrocellulose membranes) having a polymeric hydrophilic coating bonded, typically permanently bonded, to the porous membranes (e.g., nitrocellulose membranes) are further provided. In some embodiments, a polymeric hydrophilic coating is bonded onto a porous membrane such as a nitrocellulose membrane by providing an unmodified porous membrane (e.g., nitrocellulose membrane), immersing the membrane in an aqueous solution of a hydrophilic compound, and exposing the membrane to e-beam radiation, thereby polymerizing the hydrophilic coating on the porous membrane (e.g., nitrocellulose membrane). For example, a nitrocellulose membrane is immersed in an aqueous solution of a hydrophilic compound (e.g., a PEG moiety such as PEGMA, a PEGDA, or a TMPET) and then subjected to e-beam irradiation. Alternatively, in other aspects of the invention, the modified porous membranes are prepared by first subjecting a porous membrane (e.g., nitrocellulose membrane) to e-beam irradiation followed by immersing the membrane in an aqueous solution of a hydrophilic compound such as PEGMA, a PEGDA, or a TMPET. The methods of production of the modified porous membrane (e.g., nitrocellulose membrane) substrates described herein that vary, for example, in the ordering of the method steps of immersing and the e-beam irradiation step are encompassed by the instant disclosure.

When used in the context of a method for preparing a modified porous membrane as described in greater detail below, the term “immersing” the porous membrane in an aqueous solution of, for example, a hydrophilic compound such as a PEG moiety, particularly a PEGMA, a PEGDA, or a TMPET, as recited in the claims, is generally accomplished by dipping the entire porous membrane, more specifically the nitrocellulose membrane, in the aqueous solution of the hydrophilic compound (e.g., a PEG moiety, including but not limited to, a PEGMA, a PEGDA, or a TMPET and then removing any excess solution.

While nitrocellulose membranes are recited throughout the instant application, various porous membranes are encompassed by this disclosure. For exemplary purposes only and without any limitation intended, the unmodified porous membrane may include a cellulose membrane, a cellulose acetate membrane, a regenerated cellulose membrane, a nitrocellulose mixed ester membrane, a polyethersulfone membrane, a nylon membrane, a polyolefin membrane, a polyester membrane, a polycarbonate membrane, a polypropylene membrane, a polyvinylidene difluoride membrane, a polyethylene membrane, a polystyrene membrane, a polyurethane membrane, a polyphenylene oxide membrane, a poly(tetrafluoroethylene-co-hexafluoropropylene membrane, or any combination of two or more of the above porous membranes.

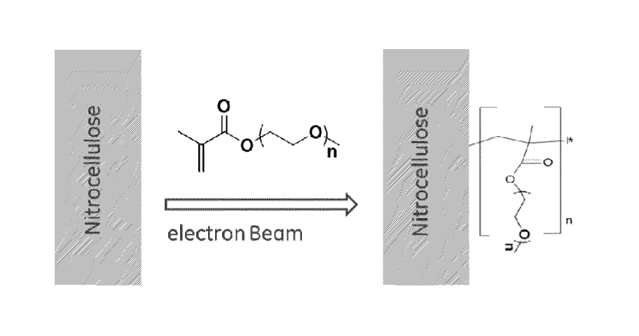

Nitrocellulose membranes are currently widely used in a variety of biological applications that require the immobilization of a particular biomolecule (e.g., DNA, RNA, or a protein such as an antibody) on a solid phase material. “Porous membrane” is intended to refer to, without limitation, to any porous membrane, including any commercially available or non-commercially available porous membrane, particularly a nitrocellulose membrane, more particularly a commercially available nitrocellulose membrane. In certain aspects of this disclosure, a nitrocellulose membrane is chemically modified to comprise, as set forth in FIG. 1, a polymeric hydrophilic coating of a PEG, wherein the polymeric hydrophilic coating decreases non-specific binding to the nitrocellulose membranes. Nitrocellulose membranes, which are made of a nitrocellulose polymer, have a strong affinity for DNA, RNA, and protein and prevent the denaturation of such biomolecules.

“Nitrocellulose membranes” as used in this application include all of those porous membrane products containing any nitrogen concentration, a diversity of pore sizes, and variable membrane thicknesses. In particular embodiments, the pore size of the porous membrane may be in the range of 0.01 to 50 microns. Moreover, pore diameter may be uniform throughout the porous membrane or, alternatively, pore diameter may be irregular. It is well within the skill and the knowledge of one in the art to select a nitrocellulose membrane, with the appropriate nitrogen content, pore size, and membrane thickness to achieve a specific, desired result. Moreover, the skilled artisan would immediately understand and appreciate the meaning of the phrase a “nitrocellulose membrane” and that such nitrocellulose membranes, include, for example, commercially available nitrocellulose membranes, may be “unbacked” membranes or alternatively contain a “backing material” or “backing support” such as a polyester (PE). The choice as to whether to use an “unbacked” or “backed” porous membrane (e.g., nitrocellulose membrane) is dependent upon the particular application to be performed and is well within the purview of one of ordinary skill in the art to make such a selection.

Nitrocellulose membranes having any nitrogen concentration, pore size, or the presence or absence of a backing support are all encompassed in the term “nitrocellulose membrane” as used herein. Nitrocellulose membranes have a variety of chemical and physical properties and are routinely used in biological techniques that require, for example, the immobilization of a biomolecule of interest (e.g., DNA, RNA, or a protein such as an antibody) to a porous membrane (e.g., nitrocellulose membrane) or for the collection of biomolecules on such membranes in order to separate them from other proteins, nucleic acids, and biomolecules or the like in a biological sample to be analyzed. Any nitrocellulose membrane may be utilized in the present disclosure.

Although porous membranes are referred to throughout the instant application, the compositions, methods of preparation, and methods of use are equally applicable to other solid phase materials useful in the immobilization of a biomolecule, as recited in the claims herein. Such solid phase materials include but are not limited to glass beads, glass fibres, latex beads, nodes, cakes, nanoparticles, hollow membrane tubes, and any combination of two or more of the above solid phase materials.

The term “biological sample” includes but is not limited to blood, serum, lymph, saliva, mucus, urine, other bodily secretions, cells, and tissue sections obtained from a human or non-human organism. Biological samples may be obtained by an individual undergoing the diagnostic test herself (e.g., blood glucose monitoring) or by a trained medical professional through a variety of techniques including, for example, aspirating blood using a needle or scraping or swabbing a particular area, such as a lesion on a patient's skin. Methods for collecting various biological samples are well known in the art.

“Immunoassay” is used herein in its broadest sense to include any technique based on the interaction between an antibody and its corresponding antigen. Such assays are based on the unique ability of an antibody to bind with high specificity to one or a very limited group of similar molecules (e.g., antigens). A molecule that binds to an antibody is called an antigen. Immunoassays can be carried out using either the antigen or antibody as the “capture” molecule to “entrap” the other member of the antibody-antigen pairing.

An exemplary, albeit not exhaustive list of immunoassays includes a lateral flow assay (e.g., a home pregnancy test), a radioimmunoassay (RIA), an enzyme immunoassay (EIA), an enzyme-linked immunosorbent assay (ELISA), a fluorescent immunoassay, and a chemiluminescent immunoassay. The skilled artisan in the field possesses the skills needed to select and implement the appropriate method(s) for a particular situation, as well as the techniques for performing these immunoassays, as well as the skills to interpret the results. Immunoassays may produce qualitative or quantitative results depending on the particular method of detection selected.

The lateral flow assay is a common immunoassay, largely due to its ease of use, and includes such products as commercially available home-pregnancy tests and routine drug tests. Lateral flow assays are particularly advantageous because the devices and methods are generally simple to use and to interpret the test results, even by an individual with no formal medical training. Lateral flow devices and methods are intended to detect the presence or absence of a target analyte or biomolecule (e.g., human chorionic gonadotropin (hCG) in a lateral flow home pregnancy test) in a biological sample (e.g., urine). Although there is variation among lateral flow devices and assays, these tests are commonly used for home testing, point of care testing, and laboratory use. Lateral flow assays are often presented in a convenient “dipstick” format, as described in the examples below, in which the biological sample to be tested flows along a solid substrate (e.g., a porous membrane, often a nitrocellulose membrane) via capillary action. In certain formats of lateral flow assays, the dipstick is immersed in the biological sample, it encounters one or more reagents previously imprinted on the dipstick as the biological sample flows up the test strip, thereby encountering lines or zones on the test strip that have been previously imprinted with, for example, an antibody or antigen (e.g., hCG). When the biological sample encounters this reagent(s), a signal is generated to indicate whether the test is positive or negative for the presence of the analyte or biomolecule of interest (e.g., frequently a line visible to the naked eye as in the detection of hCG in a home pregnancy test indicative of the presence of hCG in the patient's urine).

Lateral flow devices and methods are well known in the art. See, for example, U.S. Pat. Nos. 4,094,647; 4,313,734; 4,857,453; 5,073,484; 5,559,041; 5,571,726; 5,578,577; 5,591,645; 6,187,598; 6,352,862; and 6,485,982; all of which are herein incorporated by reference in their entirety. Inclusion of a modified porous membrane of this disclosure, such as a modified nitrocellulose membrane comprising a polymeric coating of GMA polymers, in known, for example, lateral flow devices and assays would significantly improve the performance, sensitivity, and specificity of such lateral flows devices and immunoassays, decrease the concentration of the analyte or biomolecule needed to obtain an accurate test results, and reduce the time to detect the presence or absence of the analyte or biomolecule, thereby minimizing the time required to acquire the test result.

All antibodies are proteins, more specifically glycoproteins, and exhibit binding specificity to an antigen (e.g., a portion of a polypeptide) of interest. The term “antibody” is used in the broadest sense and covers fully assembled antibodies, antibody fragments that can bind antigen (e.g., Fab′, F′(ab)₂, Fv, single chain antibodies, diabodies), and recombinant peptides comprising the foregoing. “Antibody fragments” comprise a portion of an intact antibody, preferably the antigen-binding or variable region of the intact antibody. Examples of antibody fragments include Fab, Fab′, F(ab′)₂, and Fv fragments, diabodies, and linear antibodies (Zapata et al. (1995) Protein Eng. 8(10):1057 1062), single-chain antibody molecules, and multi-specific antibodies formed from antibody fragments. Any antibody or antibody fragment may be used in the practice of the invention.

In certain aspects of this invention, detection of antibody binding or immobilization on a solid phase material, including but not limited to a nitrocellulose membrane, is needed. Any method known in the art for detecting antibody binding to a nitrocellulose membrane is encompassed by the disclosed invention. The determination and optimization of appropriate antibody binding detection techniques is standard and well within the routine capabilities of one of skill in the art. In some embodiments, detection of antibody binding can be facilitated by coupling the antibody to a detectable substance. Examples of detectable substances include various enzymes, prosthetic groups, fluorescent materials, luminescent materials, bioluminescent materials, and radioactive materials. Exemplary suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin; a detectable luminescent material that may be couple to an antibody includes but is not limited to luminol; examples of bioluminescent materials include luciferase, luciferin, and aequorin; and examples of suitable radioactive material for detection of antibody binding include ¹²⁵I, ¹³¹I, ³⁵S, or ³H.

The modified porous membranes comprising a polymer coating of, for example, a PEG, may be further modified to comprise a hydrophilic compound immobilized on the porous membrane. The introduction of a hydrophilic compound onto the modified porous membrane comprising a polymeric coating may act as a blocking agent to decrease non-specific, background binding to the porous membrane (e.g., nitrocellulose membrane). Minimizing non-specific, background binding to a porous membrane improves the signal to noise ratio in, for example, immunoassays based on the specific interaction of an antibody immobilized on the porous membrane and a specific biomolecule of interest (e.g., a protein) in a sample being analyzed for the presence or quantity of this biomolecule.

In certain aspects of the invention, the claimed modified nitrocellulose membranes, are prepared as described above using an aqueous solution of a polymeric hydrophilic coating. The solution of the polymeric hydrophilic coating may further comprise a co-solvent to improve the solubility of the hydrophilic compound in water. For example, a surfactant, more particularly a non-ionic surfactant (e.g., polyoxyethylene (20) sorbitan monolaurate (Tween-20™)), may be used as a co-solvent to increase solubility of, for example, a PEG, in water. One of skill in the art will appreciate that the appropriate amount of a particular co-solvent (e.g., a nonionic surfactant such as polyoxyethylene (20) sorbitan monolaurate (Tween-20™) needed to increase the solubility of, for example, a PEG (e.g., a PEGMA, a PEGDA, or a TMPET) must be determined and optimized experimentally.

The dosage of e-beam radiation used in the methods of grafting a polymer coating onto a porous membrane, particularly a nitrocellulose membrane, is selected to maximize the amount of the polymeric hydrophilic coating that is bonded to the nitrocellulose membrane while also limiting degradation of the porous membrane (e.g., nitrocellulose membrane) known to result from e-beam irradiation. One of skill in the art will recognize that the appropriate dose of e-beam radiation used in the preparation of the modified porous membranes of the invention will need to be optimized experimentally. In particular embodiments, the dose of e-beam radiation used in the methods to prepare a modified porous membrane may be in the range of less than 1 kGy to approximately 50 kGy. The design of assays to optimize parameters such as the amount of the polymeric hydrophilic coating, optional surfactant, and the dose of e-beam radiation appropriate for use in the methods of the invention is standard and well within the routine capabilities of those of skill in the art.

The modified porous membranes of the invention find use in various biological applications that are dependent upon the immobilization of a biomolecule on a porous membrane (e.g., a nitrocellulose membrane), including but not limited to immunoassays, in vitro diagnostic tests, and techniques for the isolation of a biomolecule of interest. Nitrocellulose membranes are of particular use in biological techniques because of their unique ability to immobilize nucleic acids (e.g., DNA and RNA) for use in Southern and Northern blots and for their binding affinity for amino acids (e.g., protein). As a result of these properties, nitrocellulose membranes are widely used as the substrate in diagnostic tests wherein antigen-antibody binding provides the test result (e.g., home pregnancy tests).

Although the ability of unmodified nitrocellulose membranes to bind biomolecules such as nucleic acids and proteins is beneficial, the modification of these membranes, decreasing non-specific binding to the nitrocellulose membrane facilitates the immobilization of biomolecules (e.g., DNA, RNA, and protein), provides significant advantages over unmodified porous membranes.

Accordingly, in certain aspects of the invention, a method is provided for improving the sensitivity of an immunoassay that uses a nitrocellulose membrane for immobilization of a biomolecule, wherein the nitrocellulose membrane used in the immunoassay is a nitrocellulose membrane comprising a polymeric hydrophilic coating bonded to the nitrocellulose membrane. Although the methods described herein can be used in the practice of any immunoassay, in certain embodiments the immunoassays include but are not limited to a lateral flow immunoassay, a radioimmunoassay, an enzyme immunoassay (EIA), an enzyme-linked immunosorbent assay (ELISA), a fluorescent immunoassay, and a chemiluminescent immunoassay.

A method for decreasing non-specific binding in an immunoassay that uses a nitrocellulose membrane for immobilization of a biomolecule, wherein the nitrocellulose membrane used in the immunoassay is a nitrocellulose membrane comprising a polymeric hydrophilic coating bonded to the nitrocellulose membrane.

In a further embodiment, a method is disclosed for decreasing non-specific binding in an immunoassay that uses a nitrocellulose membrane for immobilization of a biomolecule, wherein the nitrocellulose membrane used in the immunoassay is a nitrocellulose membrane comprising a polymeric hydrophilic coating bonded to the nitrocellulose membrane. Moreover, a method is additionally provided for decreasing signal to noise ratio in an immunoassay that uses a nitrocellulose membrane for immobilization of a biomolecule, wherein the nitrocellulose membrane used in the immunoassay is a nitrocellulose membrane comprising a polymeric hydrophilic coating bonded to the nitrocellulose membrane.

The methods described above wherein a chemically modified porous membrane (e.g., a nitrocellulose membrane) comprises a polymeric hydrophilic membrane that decreases non-specific binding to the nitrocellulose membrane and also has the ability to bind a biomolecule such as DNA, RNA, or a protein imparts a number of advantages on immunoassays that utilize these modified porous membranes. For example, such membranes improve the performance and sensitivity of numerous immunoassays by decreasing non-specific binding to the membranes and by increasing the signal to noise ratio relative to that observed in immunoassays performed with unmodified porous membranes (e.g., nitrocellulose membranes), thereby permitting a more easily “observable” distinction between a positive and negative result in, for example an in vitro diagnostic assay. Moreover, the modified porous membranes disclosed may eliminate the need for the use of other blocking agents (e.g., surfactants) that are traditionally required in other assays that rely on the immobilization of a biomolecule on a porous membrane.

A variety of immunoassays exist in the art, including those for drug testing, hormones, numerous disease-related proteins, tumor protein markers, and protein markers for cardiac injury. Immunoassays are also used to detect antigens on infectious agents such as Hemophilus, Cryptococcus, Streptococcus, Hepatitis B virus, HIV, Lyme disease, and Chlamydia trichomatis. These immunoassay tests are commonly used to identify patients with these and other diseases. Accordingly, compositions and methods for improving the sensitivity, specificity, and detection limits in immunoassays are of great importance in the field of diagnostic medicine.

The methods described herein may further permit the detection of one or more biomolecules in a biological sample (e.g., blood, serum, lymph, urine, saliva, mucus, bodily secretions, cells, or tissue). The biomolecules detected using the methods described here may be an antigen associated with a disease, such as a bacterial disease, a viral disease, or a fungal disease or a protein biomarker associated with diseases such as a cancer, a cardiac dysfunction, a heart attack, or an inflammatory disease.

The term “analyte” refers to a substance or chemical constituent whose presence or absence in, for example, a biological sample is being determined via an immunoassay or other diagnostic test.

“Biomolecule” as used herein refers without limitation to a nucleic acid (e.g., DNA or RNA) or a protein (e.g., an antibody) but further includes any organic molecule removed from an organism (e.g., a human patient).

The following examples are offered by way of illustration and not by way of limitation:

### EXAMPLES

### Example 1

### Bonding of a PEG on a Nitrocellulose Membrane

PEG derivatives, specifically PEGMA of molecular weights under of 300 and 2080 Da, polyethylene glycol diacrylate (PEGDA) of molecular weights under of 300 and 575 Da, and trimethylolpropane ethoxylate triacrylate (TMPET) of a molecular weight under 912 Da, were analyzed for their ability to bond to nitrocellulose when exposed to e-beam irradiation.

A dipping solution was prepared containing a PEG derivative monomer, with or without the non-ionic surfactant Tween-20™ (e.g., polyoxyethylene (20) sorbitan monolaurate), a non-ionic surfactant used to enhance the monomer solubility in water. The nitrocellulose membrane was immersed in an aqueous PEG solution and subjected to e-beam irradiation at a dose of 10 kGy or 50 kGy. The nitrocellulose membranes were washed with water thoroughly and then dried under vacuum overnight. Initial assessment on the grafting was measured by the weight gain of the nitrocellulose membrane following the above treatment. The results are set forth in Table 1.

TABLE 1
Factors Analyzed for Bonding of a
PEG to a Nitrocellulose Membrane

| | # | Conc. | Tween 20 | e-beam | Wt. gain (NC) |
|---|---|---|---|---|---|
| PEGMA, MW 300 | 1 | 9% | 0% | 10 kGy | 2.7% |
| | 2 | 10% | 30% | 10 kGy | 11.1% |
| PEGMA, MW 2080 | 3 | 10% | 0% | 10 kGy | 0% |
| | 4 | | | 50 kGy | 0% |
| | 5 | | 15% | 10 kGy | 0% |
| | 6 | | 30% | 10 kGy | 0% |
| PEGDA, MW 300 | 7 | 9.3% | 7.4% | 10 kGy | 19.6% |
| | 8 | 5% | 1% | 10 kGy | 6.1% |
| | 9 | 9.7% | 3.2% | 10 kGy | 35.1% |
| PEGDA, MW 575 | 10 | 5% | 2.5% | 10 kGy | 8.7% |
| | 11 | 2.5% | 0.5% | 10 kGy | 1.7% |
| TMPET, MW 912 | 12 | 9.5% | 4.3% | 10 kGy | 34.5% |

The grafting efficiency of PEGMA300 was enhanced by addition of polyoxyethylene (20) sorbitan monolaurate (Tween-20™) to the dipping solution. This increase in bonding efficiency of PEGMA300 to the nitrocellulose membrane was also confirmed by ATR-FTIR. Both PEGDA300 and PEGDA575 showed a linear relationship between grafting (e.g., bonding efficiency) and the monomer concentration used in the dipping solution. Polyoxyethylene (20) sorbitan monolaurate (Tween-20™) was used to improve solubility of the PEGDA300 and PEGDA575 at higher concentrations of the PEG monomer, with a weight gain of nitrocellulose-PEGDA membranes of up to 35.1% was achieved. TMPET 912 also showed good grafting of the PEG derivative on the nitrocellulose membrane with a 34.5% weight gain achieved. Grafting of the PEG derivatives was confirmed by IR analysis in accordance with standard methods in the art.

### Example 2

### Nitrocellulose Grafted with a PEG Exhibits Decreased Non-Specific Binding

Assays were performed to determine if a nitrocellulose membrane bound with a polymeric PEG coating. A running buffer was prepared by coating 40 nm gold nanoparticles with a final concentration of 0.2 mg/ml BSA and an optical density of 0.8

Nitrocellulose membranes modified with PEG derivatives were prepared essentially as described above in Example 1. The modified nitrocellulose membranes were dipped into the running buffer, and the gold label was used to serve as the indicator of BSA presence of on the membranes.

The gold nanoparticles aggregated in the origin of the flow on the unmodified nitrocellulose membranes, suggestive of non-specific binding of BSA to the membrane, whereas the modified nitrocellulose membranes grafted with a PEG derivative the gold solution was able to flow smoothly up the membrane, and gold aggregates were only observed on the end (e.g., terminus) of the modified nitrocellulose-PEG membranes, strongly suggesting that the modified PEG-grafted membranes were able to block non-specific interaction between a protein (e.g., BSA) and the modified membrane. Blocking of non-specific protein binding was observed when as little as 1.7% PEGDA575 was grafted on the nitrocellulose membrane. Optimal blocking of non-specific binding was observed at PEG grafting efficiency around ˜10%.

### Example 3

### PEG-Grafted Nitrocellulose Membranes Exhibit Reduced Non-Specific Binding Similar to that Observed with Blocking Agents

Modified nitrocellulose membranes grafted with a PEG (e.g., PEGMA300 or PEGDA575) were assembled into a half stick lateral flow device, with the absorbent pad laminated on top of the modified nitrocellulose membrane, with an approximately 1 mm overlap, and the lateral flow device further supported by a polyester housing material with G&L 187 glue. Unmodified nitrocellulose membranes not grafted with any PEG served as a control.

A running buffer was prepared by coating 40 nm gold nanoparticles with a final concentration of 0.2 mg/ml BSA and an optical density of 0.8 OD. For purposes of comparison, a second running buffer was prepared by coating 40 nm gold nanoparticles with a final concentration of 0.2 mg/ml BSA and further comprising a 0.5% polyoxyethylene (20) sorbitan monolaurate (Tween-20™) solution, wherein the polyoxyethylene (20) sorbitan monolaurate (Tween-20™) served as a blocking agent. The half sticks were dipped into the above described running buffers, with or without polyoxyethylene (20) sorbitan monolaurate (Tween-20™), the capillary flow of the gold particles on the nitrocellulose membranes was monitored, and the backgrounds of the membrane were then analyzed by L-a-b colorimetric analysis, where “L, a, b” indicates “lightness, redness, greenness”.

In those examples in which the running buffer contained blocking agent, all nitrocellulose membranes (modified or non-modified) were able to allow gold labeled BSA to flow smoothly from the bottom to the top, with all colors absorbed onto the absorbent pad.

When the running buffer containing the blocking agent was used, the BSA-labeled gold nanoparticles were trapped in the un-modified nitrocellulose membranes due to non-specific binding. The modified PEG-grafted nitrocellulose membranes, however, decreased non-specific binding to the membrane such that no visible color (e.g., gold) accumulated on the membranes after the BSA-labeled gold nanoparticles were allowed to completely flow through the membrane. When the color of background of the membranes was quantified, the modified PEG-nitrocellulose membranes exposed to the running containing no blocking agent (e.g., polyoxyethylene (20) sorbitan monolaurate (Tween-20™)) possessed a similar value to that observed with the unmodified nitrocellulose membrane exposed to the running buffer comprising the blocking agent polyoxyethylene (20) sorbitan monolaurate (Tween-20™). These results strongly suggest that the polymeric PEG coating on the modified nitrocellulose membranes functions in a similar and equivalent fashion to that of traditional blocking agents such as polyoxyethylene (20) sorbitan monolaurate (Tween-20™).

### Example 4

### Nitrocellulose Membranes Grafted with a PEG Decrease Non-Specific Binding

Nitrocellulose membranes were printed with a test line (1 mg/ml anti-HCG-α) and a control line (0.5 mg/ml goat-anti-mouse-IgG), similar to a standard home pregnancy test, and then assembled into a half stick lateral flow device with absorbent pad laminated on top of nitrocellulose with an approximately 1 mm overlap, and the device further supported by a polyester housing material with G&L 187 glue.

For each half stick, 100 μl of running buffer containing 600 mIU/ml human chorionic gonadotropin (hCG; a hormone elevated in pregnancy), 1.5 ng/ml 40 nm gold nanoparticles coated with 0.15 mg/ml hCG-β was prepared. For comparison purposes, a second running buffer containing the blocking agent 0.5% polyoxyethylene (20) sorbitan monolaurate (Tween-20™) was also prepared. The half sticks were dipped in the running buffers for approximately 30 minutes to allow for completion of the assay. Signal intensities of the test line were quantified using Image J and normalized against the results obtained using unmodified nitrocellulose membranes in the presence of the blocking agent polyoxyethylene (20) sorbitan monolaurate (Tween-20™).

The signal intensity observed with the modified PEG-grafted nitrocellulose membranes was comparable to that seen with the unmodified membranes were exposed to running buffers comprising the blocking agent polyoxyethylene (20) sorbitan monolaurate (Tween-20™), strongly supporting that using modified nitrocellulose membranes comprising a polymeric hydrophilic (e.g., a PEG) coating may reduce or eliminate the need to include a traditional blocking agent like blocking agent polyoxyethylene (20) sorbitan monolaurate (Tween-20™) in lateral flow assays (e.g., home pregnancy tests) while maintaining the sensitivity of the diagnostic test.

While only certain features of the invention have been illustrated and described herein, many modifications and changes will occur to those skilled in the art. It is, therefore, to be understood that the appended claims are intended to cover all such modifications and changes as fall within the true spirit of the invention.

All publications, patent publications, and patents are herein incorporated by reference to the same extent as if each individual publication or patent was specifically and individually indicated to be incorporated by reference.

1. A nitrocellulose membrane comprising a polymeric hydrophilic coating bonded to the nitrocellulose membrane.

2. The nitrocellulose membrane of claim 1, wherein the polymeric hydrophilic coating comprises a polyethylene glycol (PEG) moiety, a polyvinyl alcohol, a hydroxyl group, a negatively charged ionic group, a positively charged ionic group, a zwitterionic group, or any combination thereof.

3. The nitrocellulose membrane of claim 2, wherein the polymeric hydrophilic coating comprises a PEG moiety.

4. The nitrocellulose membrane of claim 3, wherein the PEG moiety is selected from the group consisting of a PEGMA, a PEGDA, and a TMPET.

5. The nitrocellulose membrane of claim 4, wherein a biomolecule of interest is immobilized on the nitrocellulose membrane.

6. The nitrocellulose membrane of claim 5, wherein the biomolecule of interest is a protein or a nucleic acid.

7. The nitrocellulose membrane of claim 6, wherein the biomolecule of interest is a protein.

8. The nitrocellulose membrane of claim 7, wherein the protein is an antibody.

9. The nitrocellulose membrane of claim 5, wherein the nitrocellulose membrane displays a decrease in non-specific binding relative to non-specific binding to an unmodified nitrocellulose membrane.

10. The nitrocellulose membrane of claim 9, wherein the nitrocellulose membrane further displays an increase in signal to noise ratio.

11. The nitrocellulose membrane of claim 1, wherein the polymeric hydrophilic coating is covalently linked to the nitrocellulose membrane.

12. The nitrocellulose membrane of claim 1, wherein the pore size of the nitrocellulose membrane is in the range of 0.01 to 50 microns.

13. A device for performing an immunoassay that comprises a nitrocellulose membrane comprising a polymeric hydrophilic coating bonded to the nitrocellulose membrane.

14. The device of claim 13, wherein the device is use in a lateral flow immunoassay.

15. A method for preparing a nitrocellulose membrane comprising a polymeric hydrophilic coating bonded to the nitrocellulose membrane, the method comprising:
a) providing an unmodified nitrocellulose membrane;
b) immersing the nitrocellulose membrane in an aqueous solution of a hydrophilic compound;
c) exposing the porous membrane to electron beam (e-beam) radiation; and
d) drying the nitrocellulose membrane, thereby preparing a nitrocellulose membrane comprising a polymeric hydrophilic coating bonded to the nitrocellulose membrane.

16. The method of claim 15, wherein the aqueous solution of the hydrophilic compound further comprises a non-ionic surfactant.

17. The method of claim 16, wherein the non-ionic surfactant is polyoxyethylene (20) sorbitan monolaurate.

18. A method for improving the sensitivity of an immunoassay that uses a nitrocellulose membrane for immobilization of a biomolecule, wherein the nitrocellulose membrane used in the immunoassay is a nitrocellulose membrane comprising a polymeric hydrophilic coating bonded to the nitrocellulose membrane.

19. The method of claim 18, wherein the immunoassay is selected from the group consisting of a lateral flow immunoassay, a radioimmunoassay, an enzyme immunoassay (EIA), an enzyme-linked immunosorbent assay (ELISA), a fluorescent immunoassay, and a chemiluminescent immunoassay.

20. The method of claim 19, wherein the method permits the detection of one or more biomolecules in a biological sample.

21. The method of claim 20, wherein the biological sample is blood, serum, lymph, urine, saliva, mucus, bodily secretions, cells, or tissue.

22. The method of claim 21, wherein the biomolecule is an antigen associated with a disease.

23. The method of claim 22, wherein the disease is a bacterial disease, a viral disease, or a fungal disease.

24. A method for decreasing non-specific binding in an immunoassay that uses a nitrocellulose membrane for immobilization of a biomolecule, wherein the nitrocellulose membrane used in the immunoassay is a nitrocellulose membrane comprising a polymeric hydrophilic coating bonded to the nitrocellulose membrane.

25. A method for decreasing signal to noise ratio in an immunoassay that uses a nitrocellulose membrane for immobilization of a biomolecule, wherein the nitrocellulose membrane used in the immunoassay is a nitrocellulose membrane comprising a polymeric hydrophilic coating bonded to the nitrocellulose membrane.
